(19) United States
(12) Patent Application Publication
(10) Pub. No.: US 2012/0283274 A1
(43) Pub. Date: Nov. 8, 2012

(54) CRYSTALLINE FORMS OF SUBSTITUTED PYRAZOLOPYRIMIDINES

(76) Inventors: Patrick Plitt, Bad Homburg (DE); Bernhard Hauptmeier, Gelnhausen (DE); Peter Boderke, Schwalbach (DE)

(21) Appl. No.: 13/508,404

(22) PCT Filed: Nov. 24, 2010
(86) PCT No.: PCT/EP10/68093

§ 371 Date: Jul. 26, 2012

Related U.S. Application Data
(60) Provisional application No. 61/264,269, filed on Nov. 25, 2009.

(30) Foreign Application Priority Data
Nov. 25, 2009 (EP) 09177036.2

(51) Int. Cl. A61K 31/519; A61P 25/28; A61P 25/18; C07D 471/04

(52) U.S. Cl. 514/259.3; 544/281

(57) ABSTRACT

The present invention relates to novel co-crystals of pyrazolopyrimidines and a co-crystal former, wherein the co-crystal former is an organic carboxylic acid, preferably selected from the group of gentisic acid, succinic acid and xinafoic acid.

The present invention relates to novel crystalline forms, in particular to co-crystals of substituted pyrazolo-pyrimidines with organic acids. Of particular interest are co-crystals of 6-bromo-pyrazolo[1,5-a]pyrimidin-2-yl)-(1-methyl-3,4-dihydro-1H-isoquinolin-2-yl)-methanone (hereinafter referred to from time to time as “compound A”), and in particular of R-isomer of said compound, namely 6-bromo-pyrazolo[1,5-a]pyrimidin-2-yl)-(1(R)-methyl-3,4-dihydro-1H-isoquinolin-2-yl)-methanone and an organic carboxylic acid.

Furthermore, the invention provides with methods for the preparation of co-crystals of substituted pyrazolo-pyrimidines and in particular of 6-bromo-pyrazolo[1,5-a]pyrimidin-2-yl)-(1-methyl-3,4-dihydro-1H-isoquinolin-2-yl)-methanone, in particular of 6-bromo-pyrazolo[1,5-a]pyrimidin-2-yl)-(1(R)-methyl-3,4-dihydro-1H-isoquinolin-2-yl)-methanone, with organic mono- and dicarboxylic acids.

Typical substituted pyrazolo-pyrimidines, such as the compound 6-bromo-pyrazolo[1,5-a]pyrimidin-2-yl)-(1-methyl-3,4-dihydro-1H-isoquinolin-2-yl)-methanone, including its isomers, and methods for their preparation are described in WO 2008/015269. This document describes that the pyrazolo-pyrimidines, and in particular compound A are potent mGluR5 modulators and are useful for the prevention and treatment of acute and chronic neurological disorders, in particular CNS (central nervous system) disorders, which involve excessive glutamate induced excitation.

The chemical structure of the R-enantiomer of compound A is shown in the following:

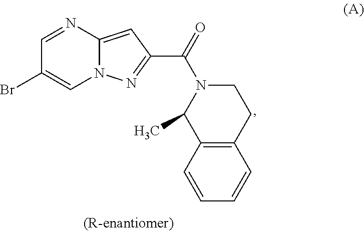

It is known that active pharmaceutical ingredients (APIs) for pharmaceutical compositions can be prepared in a variety of different forms. Most drug compounds or active pharmaceutically ingredients, such as 6-bromo-pyrazolo[1,5-a]pyrimidin-2-yl)-(1-methyl-3,4-dihydro-1H-isoquinolin-2-yl)-methanone, are dosed as solids. Many solid APIs exist in one or several crystalline forms. Frequently, the API does not crystallize on its own or it crystallizes into a crystalline form that possesses disadvantageous physical and biopharmaceutical properties. There is a need to search for alternative crystal forms in order to provide better pharmaceutical products. Good examples for particular forms include polymorphs, salts, solvates and hydrates. In addition to these established crystalline API modifications, pharmaceutical co-crystals, which can also be described as crystalline molecular complexes involving an API, have attracted the interest of chemists. The selection of a particular physical form of a pharmaceutically active ingredient represents a strategic opportunity for optimizing physical properties, such as solubility, dissolution rate, hygroscopicity, physical stability and chemical stability.

Several pharmaceutically active compounds exhibit polymorphism. Some compounds exist in more than ten different crystal form modifications. A polymorph is a solid crystalline phase of a given compound, resulting from the possibility of at least two different arrangements of the molecules of that compound in the solid state. The formation of polymorphism often depends on the crystallization conditions. Different polymorphs of a given compound possess a unique set of physicochemical properties. As a disadvantage, new polymorphic forms of a compound are normally limited to some examples.

Another known approach to obtain new crystalline forms of pharmaceutically active ingredients is the formation of hydrates and solvates. Frequently during crystallization, solvent is bound and incorporated as part of the crystal structure. Many solvents are biologically toxic and therefore, solvate-containing crystals are often avoided in the development of the solid form of a drug compound. However hydrates, wherein the crystalline structure incorporates water, are a common form of APIs and well known in pharmaceutical products. Many of the interesting pharmaceutical molecules are capable of forming hydrates. However, hydrates are often unstable and convert into anhydrous crystal forms as a result of changes in storage conditions, such as temperature, pressure or relative humidity. This conversion from hydrate to anhydrate, e.g. during storage or during the formulation process, can compromise the quality of the drug product dramatically.

The formation of salts of an API is another known approach to modify the properties of an active pharmaceutical ingredient. Salt formation can be described as an acid-base reaction between the API, which exhibits basic and/or acidic functional groups, and an acidic or basic substance. Salts of a drug compound comprise an ionic form of an API molecule in the crystal lattice. Salt formation is an attractive method to obtain novel crystalline forms of an API, because many pharmaceutical compounds exhibit either acidic or basic functionality. The widespread use of salts is evidenced by the large number of marketed crystalline salts of drug compounds.

Although co-crystals were discovered long time ago, pharmaceutical co-crystals are still an interesting goal of pharmaceutical development and optimization, in particular for heterocyclic drug compounds such as substituted pyrazolo-pyrimidines. A co-crystal according to the present invention can be understood as a crystalline complex of two or more neutral molecular compounds bound together in the crystal lattice, through non-covalent interactions, often including hydrogen bonding. Normally, no proton transfer between API and the further molecular compound (co-crystal former or counter molecule) takes place. The application of co-crystallization techniques according to the present invention provides several advantages as compared with salt formation. In principle, all types of molecules can form co-crystals, including weakly ionisable and non-ionisable compounds, which are traditionally considered to present a higher risk in terms of physical property optimization because they have either limited or no capacity for salt formation.

In 2002, the co-crystallization of the analgesic drug paracetamol with six different counter-molecules was published, each of which was capable of acting as a hydrogen-bond acceptor. Shortly thereafter, co-crystals of the drug compounds ibuprofen, flurbiprofen and aspirin with several hydrogen-bond acceptors were described. These examples showed that a series of co-crystals with common hydrogen-bonding features may be obtained. Aside from melting point data, these reports focused essentially on structural features without addressing the functional and pharmacological properties of these co-crystals.

A further advantage of the co-crystals according to the invention is that, whereas only few acidic or basic counter-ions come into consideration in a salt screen, there are several potential co-crystal forming agents (also referred to as co-crystal formers or counter-molecules) which may be used in the preparation of co-crystal with the pyrazolo-pyrimidines. Potential agents can be selected for example from the list of substances “generally recognized as safe” by the U.S. Food and Drug Administration. The increased scope of co-crystals is a benefit in suggesting a greater likelihood of achieving a desirable physical property profile for the drug, but it also presents a considerable difficulty in terms of screening efforts. Co-crystal screenings, in particular high-throughput screening methods, including improved rational co-crystal design and more efficient co-crystal screening protocols are important tools in the development of new crystalline forms. Several general methods for preparation of co-crystals are described in the literature. There are known examples of using co-crystals to enhance specific physical properties. Methods for the preparation of co-crystals include common crystallisation techniques and also more specific methods such as solid-state grinding.

The formation of co-crystals has been studied before in research, and various important studies aimed at understanding co-crystal design. In early studies several “hydrogen-bound rules,” were proposed including the observations that good proton donors and acceptors are used in hydrogen bonding, and that the best donor typically pairs with the best acceptor in a given crystal structure. The combined use of the hydrogen-bound rules with a geometric analysis was used for implementing rational co-crystal design in the synthesis of many new supramolecular structures.

Many pyrazolo-pyrimidines as described in WO 2008/015269, and in particular the compound A exhibit basic functional groups. Compound A, due to the low base capacity, shows a low pKa value of about −1.97 (calculated with correlation to pyrazolo[1,5-a]pyrimidine). Furthermore, the compound A is poorly soluble in water or aqueous solvents (below 10 μg/mL). Due to the physicochemical properties, compound A exhibits some disadvantageous pharmaceutical properties (e.g. not a perfect bioavailability). As described above, the compound A does not easily form salts with mineral acids or only instable salts, because the pKa difference between the partners is not sufficiently large. Therefore, the formation of salt is a difficult way to improve the pharmaceutical properties of compound A.

There is a high need for improved crystalline forms of (6-bromo-pyrazolo[1,5-a]pyrimidin-2-yl)-(1-methyl-3,4-dihydro-1,1-isoquinolin-2-yl)-methanone, and in particular of 6-bromo-pyrazolo[1,5-a]pyrimidin-2-yl)-(1(R)-methyl-3,4-dihydro-1H-isoquinolin-2-yl)-methanone, for the preparation of pharmaceutical compositions which exhibit enhanced solubility and dissolution characteristics and better storage stability properties.

One object of the present invention is to provide an improved crystalline form, in particular a co-crystal, of pyrazolo-pyrimidines, and in particular 6-bromo-pyrazolo[1,5-a]pyrimidin-2-yl)-(1-methyl-3,4-dihydro-1H-isoquinolin-2-yl)-methanone, particularly of 6-bromo-pyrazolo[1,5-a]pyrimidin-2-yl)-(1(R)-methyl-3,4-dihydro-1H-isoquinolin-2-yl)-methanone, and a suitable co-crystal forming agent. The novel co-crystals, preferably of compound A exhibit improved pharmaceutical properties and good storage stability (e.g. higher solubility in water, no or little hygroscopicity).

It was surprisingly found that the drug compound (6-bromo-pyrazolo[1,5-a]pyrimidin-2-yl)-(1-methyl-3,4-dihydro-1H-isoquinolin-2-yl)-methanone, and in particularly the R-isomer, form stable co-crystals with specific organic carboxylic acids. It was further found that many organic carboxylic acids and amino acids do not form co-crystals with 6-bromo-pyrazolo[1,5-a]pyrimidin-2-yl)-(1-methyl-3,4-dihydro-1H-isoquinolin-2-yl)-methanone and its optical isomers using common crystallization procedures. Some examples are benzoic acid, malic acid (1-hydroxy butanedioic acid), mandelic acid (2-hydroxy-2-phenylacetic acid), D/L tartaric acid (2,3-dihydroxy butanedioic acid), vanillic acid (4-hydroxy-3-methoxybenzoic acid), or L-aspartic acid (2-aminobutanedioic acid).

The present invention is directed to co-crystals of pyrazolo-pyrimidines, and in particular (6-bromo-pyrazolo[1,5-a]pyrimidin-2-yl)-(1-methyl-3,4-dihydro-1H-isoquinolin-2-yl)-methanone, and in particular of (6-bromo-pyrazolo[1,5-a]pyrimidin-2-yl)-(1(R)-methyl-3,4-dihydro-1H-isoquinolin-2-yl)-methanone, and at least one co-crystal former as described in the following, preferably one, two or three co-crystal former(s).

The present invention is directed to co-crystals of pyrazolo-pyrimidines, and in particular (6-bromo-pyrazolo[1,5-a]pyrimidin-2-yl)-(1-methyl-3,4-dihydro-1H-isoquinolin-2-yl)-methanone, and in particular of (6-bromo-pyrazolo[1,5-a]pyrimidin-2-yl)-(1(R)-methyl-3,4-dihydro-1H-isoquinolin-2-yl)-methanone, and a co-crystal former, wherein the co-crystal former is a carboxylic acid of general formula I

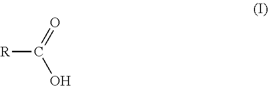

wherein R denotes:

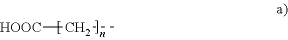

where n is 1,2,3, or 4,

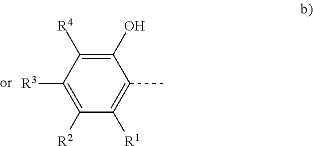

where
R¹ and R² are independently from each other hydrogen, hydroxyl or carboxyl,
R³ and R⁴ are independently from each other hydrogen, hydroxyl or carboxyl,
or R³ and R⁴, together with the carbon atoms carrying them, form an aromatic six-membered ring which may be substituted by one to four groups selected from C₁-C₅ alkyl, hydroxyl, and carboxyl.

In a preferred embodiment the present invention relates to a co-crystal of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone, and a co-crystal former, wherein the co-crystal former is a carboxylic acid of general formula I

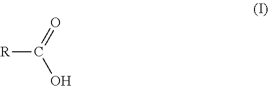

wherein R denotes:

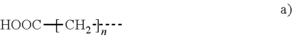

where n is 2 or 3,

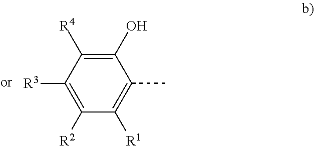

where
R¹ and R² are independently from each other hydrogen or hydroxyl,
R³ and R⁴ are hydrogen,
or R³ and R⁴, together with the carbon atoms carrying them, form an unsubstituted aromatic six-membered ring, preferably with six carbon atoms.

Preferably, the carboxylic acid co-crystal former may comprise at least two hydrogen donator groups, one selected from hydroxyl and one selected from carboxyl group, wherein a formation of preferred strong hydrogen-bonded bimolecular ring motifs could be possible.

Furthermore, it was found that 6-bromo-pyrazolo[1,5-a]pyrimidin-2-yl)-(1(R)-methyl-3,4-dihydro-1H-isoquinolin-2-yl)-methanone forms stable co-crystals with at least one of the carboxylic acids selected from the group comprising succinic acid, gentisic acid, and xinafoic acid. Moreover, it was found that the co-crystals of succinic acid, gentisic acid, and xinafoic acid have particular advantageous properties, e.g. they are not hygroscopic or less hygroscopic than compound A itself. All co-crystals mentioned above exhibit a better solubility in water than the free drug compound.

“Co-crystal” in term of the present invention means a crystalline complex of two or more neutral molecular compounds which are solids at room temperature (20-25° C.) bound together in the crystal lattice through non-covalent interactions, often including hydrogen bonding, pi-stacking, guest-host complexation, van der Waals interactions and the like. In particular said non-covalent interactions include hydrogen bonding. Hydrogen bonding may e.g. result in the formation of different intermolecular structures, such as dimers, linear chains, or cyclic structures. Each of the co-crystals exhibits distinctive physical characteristics, such as structure (e.g. characterised by PXRD pattern), melting point, heat of fusion and can be characterised inter alia thereby.

The co-crystals according to the present invention comprise (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone, and a co-crystal former that is presumably H-bonded to the compound. Other interaction as mentioned above may also play a role in formation of a co-crystal according to the present invention.

Salts and solvates of the compound A that do not further comprise a co-crystal former are not considered as co-crystals according to the present invention. However, the co-crystals according to the present invention may include one or more solvate molecules in the crystalline lattice. Thus, solvates of co-crystals or a co-crystal further comprising a compound that is a liquid at room temperature are included in the broader scope of the present invention. The co-crystals according to the present invention may also be a co-crystal of a salt of compound A and a co-crystal former, but compound A and the co-crystal former are constructed or bonded together, preferably via hydrogen bonding. The co-crystal former may be bonded directly to the compound A or may be bonded to an additional molecule (e.g. a solvate molecule) which is bound to compound A. As outlined above co-crystals in terms of the present invention can be distinguished from characteristics of classical salts and solvates/hydrates.

The novel co-crystals of (6-bromo-pyrazolo[1,5-a]pyrimidin-2-yl)-(1(R)-methyl-3,4-dihydro-1H-isoquinolin-2-yl)-methanone described in the present invention can not be thought of as classical salts due to the pKa values. Furthermore, the experimental data confirm that the crystalline compounds according to the present invention are co-crystals.

Advantageous properties of the co-crystals described in the present invention are e.g. good solubility in water, higher dissolution rate, low or no hygroscopic properties and good storability in comparison to the free compound A.

The present invention relates to a co-crystal of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-1-isoquinolin-2-yl)-methanone and at least one co-crystal former, wherein the co-crystal former preferably is a carboxylic acid selected from the group consisting of succinic acid (butanedioic acid), gentisic acid (2,5-dihydroxybenzoic acid), and xinafoic acid (1-hydroxy-2-naphthoic acid).

The present invention relates to a co-crystal of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone and a co-crystal former, wherein the co-crystal former preferably is a carboxylic acid selected from the group consisting of succinic acid (butanedioic acid), gentisic acid (2,5-dihydroxybenzoic acid), and xinafoic acid (1-hydroxy-2-naphthoic acid).

In particular the invention relates to a co-crystal of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone and a co-crystal former, wherein the molar ratio of heterocycle (compound A): co-crystal former is in the range from 1:0.1 to 1:10, preferably in the range of 1:1 to 1:10, preferably in the range from 1:1 to 1:5, more preferably about 1:1. The co-crystal is preferably a crystalline co-crystal.

In a further aspect, the present invention provides a co-crystal of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone and succinic acid, characterised by the selection of at least two, preferably at least three, more preferably at least four, even more preferably at least five powder X-ray diffraction (PXRD) peaks selected from the group consisting of 9.3, 16.0, 20.0, 22.9, and 26.0 degrees two-theta (° 2θ)+/−0.3 degrees two-theta (° 2θ). In another embodiment, the co-crystal of (6-bromo-pyrazolo[1,5-a]pyrimidin-2-yl)-(1(R)-methyl-3,4-dihydro-1H-isoquinolin-2-yl)-methanone and succinic acid can be characterised by a PXRD pattern substantially according to FIG. 1.

In a further embodiment, the co-crystal of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone and succinic acid has a DSC (differential scanning calorimetry) with a characterising melting peak at about 156.9° C. In a further embodiment, the co-crystal of (6-bromo-pyrazolo[1,5-a]pyrimidin-2-yl)-(1(R)-methyl-3,4-dihydro-1H-isoquinolin-2-yl)-methanone and succinic acid can be characterised by a DSC (differential scanning calorimetry) diagram substantially according to FIG. 2.

The present invention also provides with a co-crystal of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone and gentisic acid (2,5-dihydroxybenzoic acid), characterised by the selection of at least four, preferably at least five, more preferably at least six, even more preferably seven powder X-ray diffraction (PXRD) peaks selected from the group consisting of 6.0, 7.0, 14.0, 17.6, 21.0, 23.4, and 27.2 degrees two-theta (° 2θ)+/−0.3 degrees two-theta (° 2θ). In another embodiment the co-crystal of (6-bromo-pyrazolo[1,5-a]pyrimidin-2-yl)-(1(R)-methyl-3,4-dihydro-1H-isoquinolin-2-yl)-methanone and gentisic acid (2,5-dihydroxybenzoic acid) can be characterised by a PXRD pattern substantially according to FIG. 3.

The present invention also provides with a second polymorphic form of gentisic acid co-crystals. Thus, the present invention relates to of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone and gentisic acid (2,5-dihydroxybenzoic acid), characterised by the selection of at least one, preferably at least two, more preferably at least three, even more preferably four powder X-ray diffraction (PXRD) peaks selected from the group consisting of 6.9, 12.6, 21.2, and 27.5 degrees two-theta (° 2θ)+/−0.3 degrees two-theta (° 2θ). In another embodiment the co-crystal of (6-bromo-pyrazolo[1,5-a]pyrimidin-2-yl)-(1(R)-methyl-3,4-dihydro-1H-isoquinolin-2-yl)-methanone and gentisic acid (2,5-dihydroxybenzoic acid) can be characterised by a PXRD pattern substantially according to FIG. 4.

In another embodiment, the co-crystal of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone and gentisic acid (2,5-dihydroxybenzoic acid) can be characterised by a DSC (differential scanning calorimetry) with a characteristic melting peak at about 147.4° C. The co-crystal of (6-bromo-pyrazolo[1,5-a]pyrimidin-2-yl)-(1(R)-methyl-3,4-dihydro-1H-isoquinolin-2-yl)-methanone and gentisic acid (2,5-dihydroxybenzoic acid preferably) can be characterised by a DSC (differential scanning calorimetry) diagram substantially according to FIG. 5.

The present invention also provides with a co-crystal of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone and xinafoic acid (1-hydroxy-2-naphthalene-2-carboxylic acid; 1-hydroxy-2-naphthoic acid), characterised by the selection of at least one, preferably at least two, more preferably at least three, even more preferably four powder X-ray diffraction (PXRD) peaks selected from the group consisting of 3.9, 11.6, 18.1, and 27.2 degrees two-theta (° 2θ)+/−0.3 degrees two-theta (° 2θ). The co-crystal of (6-bromo-pyrazolo[1,5-a]pyrimidin-2-yl)-(1(R)-methyl-3,4-dihydro-1H-isoquinolin-2-yl)-methanone and xinafoic acid (1-hydroxy-2-naphthalene-2-carboxylic acid; 1-hydroxy-2-naphthoic acid) preferably can be characterised by a PXRD pattern substantially according to FIG. 6.

In a further embodiment of the invention, the co-crystal of (6-bromo-pyrazolo[1,5-a]pyrimidin-2-yl)-(1-methyl-3,4-dihydro-1H-1-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone and xinafoic acid (1-hydroxy-2-naphthalene-2-carboxylic acid; 1-hydroxy-2-naphthoic acid) can be characterised by a DSC (differential scanning calorimetry) with a characterising melting peak at about 139.2° C. The co-crystal of (6-bromo-pyrazolo[1,5-a]pyrimidin-2-yl)-(1(R)-methyl-3,4-dihydro-1H-isoquinolin-2-yl)-methanone and xinafoic acid (1-hydroxy-2-naphthalene-2-carboxylic acid; 1-hydroxy-2-naphthoic acid) can be characterised by a DSC (differential scanning calorimetry) diagram substantially according to FIG. 7.

Each co-crystal may be characterised by one or more of the above described physical properties (PXRD peaks, DSC peaks). Thus, the present invention is related to co-crystal of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone and a co-crystal former, preferably selected from gentisic acid, succinic acid, xinafoic acid which is characterised by one or more of above described physical data.

The present invention further relates to a method for the preparation of a co-crystal of (6-bromo-pyrazolo[1,5-a]pyrimidin-2-yl)-(1(R)-methyl-3,4-dihydro-1H-isoquinolin-2-yl)-methanone and a co-crystal former, wherein the co-crystal former is a carboxylic acid of general formula 1

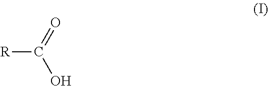

wherein R denotes:

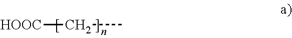

where n is 1,2,3, or 4,

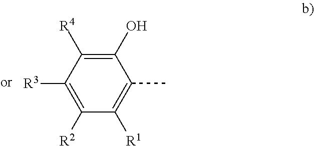

where
R¹ and R² are independently from each other hydrogen, hydroxyl or carboxyl,
R³ and R⁴ are independently from each other hydrogen, hydroxyl or carboxyl,
or R³ and R⁴, together with the carbon atoms carrying them, form an aromatic six-membered ring which may be substituted by one to four groups selected from C₁-C₅ alkyl, hydroxyl, and carboxyl,
   comprising the following steps:
a) dissolving (6-bromo-pyrazolo[1,5-a]pyrimidin-2-yl)-(1-methyl-3,4-dihydro-1H-isoquinolin-2-yl)-methanone, preferably 6-bromo-pyrazolo[1,5-a]pyrimidin-2-yl)-(1(R)-methyl-3,4-dihydro-1H-isoquinolin-2-yl)-methanone and the co-crystal former in a solvent S1,
b) evaporation of solvent S1,
c) optionally dispersing the residue obtained in step b) in a solvent S2 for at least 10 h, preferably at least 15 h, more preferably at least 24 h in a slurry.

The present invention further relates to a method for the preparation of a co-crystal of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone and a co-crystal former, wherein the co-crystal former is a carboxylic acid as defined above, preferably selected from gentisic acid, succinic acid, xinafoic acid, comprising (or consisting of) the following steps:
a) dissolving (6-bromo-pyrazolo[1,5-a]pyrimidin-2-yl)-(1-methyl-3,4-dihydro-1H-isoquinolin-2-A-methanone, preferably (6-bromo-pyrazolo[1,5-a]pyrimidin-2-yl)-(1(R)-methyl-3,4-dihydro-1H-isoquinolin-2-yl)-methanone and the co-crystal former in a solvent S1,
b) evaporation of solvent S1,
c) optionally dispersing the residue obtained in step b) in a solvent S2 (slurry) for at least 10 h, preferably at least 15 h, more preferably at least 24 h, under continuous stirring (phase equilibration).

In a preferred embodiment, the method for preparation of co-crystals of compound A and a co-crystal former selected from succinic acid and xinafoic acid comprises the phase equilibration step c).

In one embodiment of the invention, the dissolving step a) is carried out at room temperature (20-25° C.) and under normal pressure (1013.25 hPa) for a time period of 1 to 60 minutes. In another preferred embodiment the dissolving step a) is carried out under increased temperature in the range of 25° C. to 100° C.

Step a) can be carried out by first dissolving compound A in the solvent S1, and following adding the co-crystal former to the solution. Furthermore, in another embodiment, the compound A and the co-crystal former may be first mixed as solids and then dissolved in the solvent S1. As a further embodiment, step a) can be carried out by mixing of a solution of compound A in a solvent S1 and a solution of co-crystal former in a solvent S1 wherein the solvents for dissolution of compound A and co-crystal former may be different or equal. Preferably the solvents used for dissolution of compound A and co-crystal former are equal.

In a preferred embodiment, the evaporation (step b) is carried out under room temperature (20-25° C.) and under normal pressure (1013.25 hPa). Often, the step b) is carried out under air or under nitrogen flow, optionally with flow control. Optionally, the evaporation of solvent S1 (step b)) can be carried out under reduced pressure.

The phase equilibration step c) is often carried out under room temperature (20-25° C.), in another embodiment, step c) is carried out under increased or decreased temperature in the range of 0° C. to 100° C.

In one embodiment of the invention, carboxylic acid co-crystal former and the compound A are used in the method according to the present invention in a molar ratio in the range of 0.1 to 10, preferably in a molar ratio about 1:1. In a further embodiment, the carboxylic acid co-crystal former is used in a molar excess of 1.1 to 10 in relation to compound A. In a further embodiment of the invention the carboxylic acid co-crystal former is used in a molar ratio of 0.1 to 0.95 in relation to compound A. Preferably, the succinic acid is used in a molar ratio in the range of 1 to 7.5 in relation to compound A, more preferred in a molar ratio of about 1.2:1.

The drug compound (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone, and the carboxylic acid co-crystal former are often dissolved in an equimolar ratio in solvent S1 (step a).

The present invention relates to a co-crystal as described about, wherein the molar ratio of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone, and the carboxylic acid co-crystal former is in the range from 1:0.1 to 1:10, preferably in the range of 1:1 to 1:10, preferably in the range from 1:1 to 1:5. In a more preferred embodiment the molar ratio of the molar ratio of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone, and the carboxylic acid co-crystal former is 1:1 as determined by ¹H-NMR spectroscopy.

Preferably the co-crystals according to the present invention are prepared by dissolving compound A and co-crystal former and then evaporating the solvent as described above. In another embodiment the co-crystals may be prepared using other common crystallization procedures. For example compound A may be co-crystallized with the carboxylic acid co-crystal former using temperature gradients in solution or the solid state. One example is the hanging drop diffusion method which is a method for the preparation of small amounts of co-crystals.

The solvents S1 and optionally S2 are preferably at least one organic solvent selected from the group consisting of acetone, 1-butanol, tert-butyl-methyl ether (TBME), dimethyl sulfoxide (DMSO), ethanol, ethyl acetate, methyl ethyl ketone (MEK), 1-propanol, 2-propanol, tetrahydrofuran (THF), acetonitrile, dichloro methane, N,N-dimethyl formamide (DMF), 1-octanol, methanol, toluene, water, isopropyl ether (IPE) and N-methyl pyrrolidone (NMP). Preferably, the organic solvent S1 is selected from acetone, ethanol, ethyl acetate, tetrahydrofuran, and isopropyl ether (IPE), more preferably from acetone, isopropyl ether (IPE) and ethyl acetate.

Solvent S1 further may be a mixture of two, three or more of the above mentioned solvents. The solvent often is a mixture of an organic solvent (as described above) with water. Typical examples are the following mixtures: ethanol:water (1:1) and tetrahydrofuran:water (1:1). In particular, in case of low solubility of the co-crystal former in the selected solvent, solvent mixtures as described above are used.

The solvent S2 is preferably selected from at least one organic or inorganic solvent of the group consisting of acetone, 1-butanol, tert-butyl-methyl ether (TBME), dimethyl sulfoxide (DMSO), ethanol, ethyl acetate, methyl ethyl ketone (MEK), 1-propanol, 2-propanol, tetrahydrofuran (THF), acetonitrile, dichloro methane, N,N-dimethyl formamide (DMF), 1-octanol, methanol, toluene, water, isopropyl ether (IPE), and N-methyl pyrrolidone (NMP). Preferably, the solvent S2 is selected from tert-butyl-methyl ether (TBME), 1-propanol, 2-propanol, toluene, water, and isopropyl ether (IPE), more preferably from isopropyl ether (IPE) and 2-propanol.

Solvent S2 further may be a mixture of two, three or more of the above mentioned solvents. Often the solvent is a mixture of an organic solvent with water, such as e.g. mixtures ethanol:water (1:1) and tetrahydrofuran:water (1:1).

A preferred embodiment of the invention is directed to a method for the preparation of a co-crystal of (6-bromo-pyrazolo[1,5-a]pyrimidin-2-yl)-(1(R)-methyl-3,4-dihydro-1H-iso quinolin-2-yl)-methanone and succinic acid, wherein the solvent S1 is 1-propanol and solvent S2 is at least one solvent selected from isopropyl ether (IPE) and 2-propanol.

A preferred embodiment is directed to a method for preparation of a co-crystal of (6-bromo-pyrazolo[1,5-a]pyrimidin-2-yl)-(1(R)-methyl-3,4-dihydro-1H-iso quinolin-2-yl)-methanone and gentisic acid (2,5-dihydroxybenzoic acid), wherein the solvent S1 is in at least one solvent selected from acetone and isopropyl ether (IPE).

A preferred embodiment is directed to a method for preparation of a co-crystal of (6-bromo-pyrazolo[1,5-a]pyrimidin-2-yl)-(1(R)-methyl-3,4-dihydro-1H-isoquinolin-2-yl)-methanone and xinafoic acid (1-hydroxy-2-naphthalene-2-carboxylic acid), wherein the solvent S1 is ethyl acetate.

In one embodiment of the invention, the method for the preparation of a co-crystal of (6-bromo-pyrazolo[1,5-a]pyrimidin-2-yl)-(1(R)-methyl-3,4-dihydro-1H-isoquinolin-2-yl)-methanone and succinic acid comprises (or consists of) the following steps:
a) dissolving (6-bromo-pyrazolo[1,5-a]pyrimidin-2-yl)-(1(R)-methyl-3,4-dihydro-1H-isoquinolin-2-yl)-methanone and succinic acid in a molar ratio in the range of 1:1 to 1:10 in 2-propanol,
b) evaporation of the 2-propanol preferably under air or under nitrogen flow at room temperature,
c) dispersing the residue obtained in step b) in at least one solvent selected from isopropyl ether (IPE) and 2-propanol for at least 10 h, preferably at least 15 h, more preferably at least 24 h, under stirring (phase equilibration).

In one embodiment of the invention the method for the preparation of a co-crystal of (6-bromo-pyrazolo[1,5-a]pyrimidin-2-yl)-(1(R)-methyl-3,4-dihydro-1H-iso quinolin-2-yl)-methanone and gentisic acid (2,5-dihydroxybenzoic acid) comprises (or consists of) the following steps:
a) dissolving (6-bromo-pyrazolo[1,5-a]pyrimidin-2-yl)-(1(R)-methyl-3,4-dihydro-1H-isoquinolin-2-yl)-methanone and gentisic acid (2,5-dihydroxybenzoic acid) in a molar ratio in the range of about 1:1 to 1:125, preferably in the range of about 1:1.2 to 1:1.25, in at least one solvent selected from acetone and isopropyl ether (IPE),
b) evaporation of acetone preferably under nitrogen flow or in air at room temperature.

In one embodiment of the invention, the method for the preparation of a co-crystal of (6-bromo-pyrazolo[1,5-a]pyrimidin-2-yl)-(1(R)-methyl-3,4-dihydro-1H-isoquinolin-2-yl)-methanone and gentisic acid (2,5-dihydroxybenzoic acid) comprises (or consists of) the following steps:
a) dissolving (6-bromo-pyrazolo[1,5-a]pyrimidin-2-yl)-(1(R)-methyl-3,4-dihydro-1H-isoquinolin-2-yl)-methanone and gentisic acid (2,5-dihydroxybenzoic acid) in a molar ratio in the range of about 1:1 to 1:1.2.25, preferably in a molar ratio of about 1:1, in acetone,
b) evaporation of acetone preferably under nitrogen flow at room temperature.

In one embodiment step a) as described above is carried out by first dissolving compound A in acetone and following adding of gentisic acid. In another embodiment step b) is carried out by mixing a solution of compound A in acetone and a solution of gentisic acid in acetone.

In one embodiment of the invention, the method for the preparation of a co-crystal of (6-bromo-pyrazolo[1,5-a]pyrimidin-2-yl)-(1(R)-methyl-3,4-dihydro-1H-isoquinolin-2-yl)-methanone and xinafoic acid (1-hydroxy-2-naphthalene-2-carboxylic acid) comprises (or consists of) the following steps:
a) dissolving (6-bromo-pyrazolo[1,5-a]pyrimidin-2-yl)-(1(R)-methyl-3,4-dihydro-1H-1-isoquinolin-2-yl)-methanone and xinafoic acid (1-hydroxy-2-naphthalene-2-carboxylic acid) in a molar ratio of about 1:1 in ethyl acetate,
b) evaporation of ethyl acetate preferably under nitrogen flow at room temperature.

Furthermore, the present invention relates to a method for the preparation of a co-crystal of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone and at least one co-crystal former as described above, wherein preferably one, two or three co-crystal formers are applied in a method as described above.

The present invention also relates to a pharmaceutical composition comprising at least one, preferably one, two or three co-crystal(s) according to the present invention.

The present invention also relates to a pharmaceutical composition comprising a co-crystal of (6-bromo-pyrazolo[1,5-a]pyrimidin-2-yl)-(1-methyl-3,4-dihydro-1H-isoquinolin-2-yl)-methanone, preferably (6-bromo-pyrazolo[1,5-a]pyrimidin-2-yl)-(1(R)-methyl-3,4-dihydro-1H-isoquinolin-2-yl)-methanone, or a pharmaceutically acceptable derivative or analog thereof and a-co crystal former according to the present invention, together with one or more pharmaceutically acceptable excipients. The types of pharmaceutical compositions, the excipients and the preparation are described in more detail in WO2008/015270 and WO 2008/015269.

The present invention also relates to a co-crystal of (6-bromo-pyrazolo[1,5-a]pyrimidin-2-yl)-(1(R)-methyl-3,4-dihydro-1H-isoquinolin-2-yl)-methanone and a co-crystal former as described in the present invention for use of treating and/or preventing a condition or disease associated with abnormal glutamate neurotransmission, preferably a condition or disease as described below.

Preferably the present invention is directed to co-crystals of (6-bromo-pyrazolo[1,5-a]pyrimidin-2-yl)-(1(R)-methyl-3,4-dihydro-1H-isoquinolin-2-yl)-methanone and a co-crystal former as described in the present invention for use of treating and/or preventing condition or disease from the following: Alzheimer's disease, positive and/or negative symptoms of schizophrenia, cognitive impairment, or for cognitive enhancement and/or neuroprotection.

The term “analog” or “derivative” is used herein in the conventional pharmaceutical sense, to refer to a molecule that structurally resembles a reference molecule, but has been modified in a targeted and controlled manner to replace one or more specific substituents of the referent molecule with an alternate substituent, thereby generating a molecule which is structurally similar to the reference molecule. In addition, using methods known to those skilled in the art, analogs and derivatives of the known compound A can be created which have improved therapeutic efficacy, i.e., higher potency and/or selectivity at a specific targeted receptor type, either greater or lower ability to penetrate mammalian blood-brain barriers (e.g., either higher or lower blood-brain barrier permeation rate), fewer side effects, etc.

The phrase “pharmaceutically acceptable”, as used in connection with compositions of the invention, refers to molecular entities and other ingredients of such compositions that are physiologically tolerable and do not typically produce untoward reactions when administered to a mammal, e.g., a human. Preferably, as used herein, the term “pharmaceutically acceptable” means approved by a regulatory agency of the Federal or a state government or listed e.g. in the U.S. Pharmacopeia or other generally recognized pharmacopoeia for use in mammals, and more particularly in humans.

Co-crystals according to the present invention may find application in the treatment and/or prophylaxis of various disorders of a living animal body, especially a human. Co-crystals also find application in the treatment of indications in a living animal body, especially a human, wherein a particular condition does not necessarily exist but wherein a particular physiological parameter may be improved through administration of the instant compounds, including cognitive enhancement.

The method-of-treating a living animal body with a co-crystal of the invention, for the inhibition of progression or alleviation of the selected ailment therein, is as previously stated possible by any normally-accepted pharmaceutical route, employing the selected dosage which is effective in the alleviation of the particular ailment desired to be alleviated. Use of the co-crystals of the present invention in the manufacture of a medicament for the treatment of a living animal for inhibition of progression or alleviation of selected ailments or conditions, particularly ailments or conditions susceptible to treatment with a Group 1 mGluR modulator is carried out in the usual manner comprising the step of admixing an effective amount of a co-crystal of the invention with a pharmaceutically-acceptable diluent, excipient, or carrier.

Representative pharmaceutical compositions may be prepared by combining the co-crystal ingredient with one or more suitable and pharmaceutically-acceptable excipients. These pharmaceutical compositions can be applied via different routes like the oral, dermal, parenteral, pulmonary, rectal, transmucosal and nasal route. Pharmaceutical dosage forms can be e.g. powders, granules, tablets, film coated tablets, modified release tablets, hard capsule, soft capsules, solutions, suspensions, emulsions, creams, ointments, gels, transdermal patches, aerosol formulation, powder formulations for inhalation and micro- or nanoparticles based formulations, thus to produce medicaments for animal and preferred human use. Examples of suitable formulation types, including a co-crystal of compound A, are given in WO 2008/015269. In a preferred embodiment co-crystals according to the present invention are used in solid dosage forms such as tablets and capsules. A suitable formulation of present co-crystals is furthermore, a suspension of co-crystals in a solvent.

In the pharmaceutical compositions of the present invention, the co-crystal according to the present invention is formulated as dosage units containing e.g. from 0.1 to 4000 mg, preferably 1 to 2000 mg, of said compound per dosage unit for daily administration. For all aspects of the invention, particularly medical ones, the administration of a compound or composition has a dosage regime, which will ultimately be determined by the attending physician and will take into consideration factors such as the compound being used, animal type, gender, age, weight, severity of symptoms, method of administration, adverse reactions and/or other contraindications.

The physiologically acceptable compound according to the invention will normally be administered in a daily dosage regimen (for an adult patient) of, for example, an oral dose of between 0.01 mg/kg (mg per kilogram of body weight of the mammal to be treated) and 100 mg/kg, preferably between 0.1 mg/kg and 75 mg/kg.

Furthermore, the invention relates to the use of a composition comprising a co-crystal of (6-bromo-pyrazolo[1,5-a]pyrimidin-2-yl)-(1-methyl-3,4-dihydro-1H-isoquinolin-2-yl)-methanone (and/or its R-enantiomer) according to the present invention as a medicament to provide neuroprotection in an animal, including a human.

Furthermore, the invention relates to the use of a co-crystal of (6-bromo-pyrazolo[1,5-a]pyrimidin-2-yl)-(1-methyl-3,4-dihydro-1H-isoquinolin-2-yl)-methanone (and/or its R-enantiomer) according to the present invention for treatment of a condition associated with abnormal glutamate neurotransmission or in which modulation of mGluR5 receptors results in therapeutic benefit.

In particular, the present invention deals with the use of a co-crystal according to the present invention for the preparation of a medicament for the prevention and/or treatment of a condition or disease selected from the following:

Alzheimer's disease, Creutzfeld-Jakob's syndrome/disease, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic clerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, trauma, hypoglycaemia, hypoxia, perinatal hypoxia, ischaemia, ischaemia resulting from cardiac arrest or stroke or bypass operations or transplants, convulsions, epileptic convulsions, epilepsy, temporal lobe epilepsy, myoclonic epilepsy, inner ear insult, inner ear insult in tinnitus, tinnitus, sound- or drug-induced inner ear insult, sound- or drug-induced tinnitus, L-dopa-induced dykinesias, L-dopa-induced dykinesias in Parkinson's disease therapy, dyskinesias, dyskinesia in Huntington's disease, drug induced dyskinesias, neuroleptic-induced dyskinesias, haloperidol-induced dyskinesias, dopaminomimetic-induced dyskinesias, chorea, Huntington's chorea, athetosis, dystonia, stereotypy, ballism; tardive dyskinesias, tic disorder, torticollis spasmodicus, blepharospasm, focal and generalized dystonia, nystagmus, hereditary cerebellar ataxias, corticobasal degeneration, tremor, essential tremor, abuse, addiction, nicotine addiction, nicotine abuse, alcohol addiction, alcohol abuse, opiate addiction, opiate abuse, cocaine addiction, cocaine abuse, amphetamine addiction, amphetamine abuse, anxiety disorders, panic disorders, anxiety and panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, depression resulting from Borna virus infection, major depression resulting from Borna virus infection, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, diabetic neuropathic pain (DNP), pain related to rheumatic arthritis, allodynia, hyperalgesia, nociceptive pain, cancer pain, posttraumatic stress disorder (PTSD), schizophrenia, positive or cognitive or negative symptoms of schizophrenia, spasticity, Tourette's syndrome, urinary incontinence, vomiting, pruritic conditions, pruritis, sleep disorders, micturition disorders, neuromuscular disorder in the lower urinary tract, gastroesophageal reflux disease (GERD), gastrointestinal dysfunction, lower esophageal sphincter (LES) disease, functional gastrointestinal disorders, dyspepsia, regurgitation, respiratory tract infection, bulimia nervosa, chronic laryngitis, asthma, reflux-related asthma, lung disease, eating disorders, obesity, obesity-related disorders, obesity abuse, food addiction, binge eating disorders, agoraphobia, generalized anxiety disorder, obsessive-compulsive disorder, panic disorder, posttraumatic stress disorder, social phobia, phobic disorders, substance-induced anxiety disorder, delusional disorder, schizoaffective disorder, schizophreniform disorder, substance-induced psychotic disorder, or delirium; inhibition of tumour cell growth, migration, invasion, adhesion and toxicity in the peripheral tissues, peripheral nervous system and CNS; neoplasia, hyperplasia, dysplasia, cancer, carcinoma, sarcoma, oral cancer, squamous cell carcinoma (SCC), oral squamous cell carcinoma (SCC), lung cancer, lung adenocarcinoma, breast cancer, prostate cancer, gastric cancer, liver cancer, colon cancer, colorectal carcinoma, rhabdomyosarcoma, brain tumour, tumour of a nerve tissue, glioma, malignant glioma, astroglioma, neuroglioma, neuroblastoma, glioblastoma, medulloblastoma, cancer of skin cells, melanoma, malignant melanoma, epithelial neoplasm, lymphoma, myeloma, Hodgkin's disease, Burkitt's lymphoma, leukemia, thymoma, and other tumours.

The disorders which can be treated have already been described above. Preferred conditions and indications which are:

a) For mGluR5 modulators: chronic pain, neuropathic pain, diabetic neuropathic pain (DNP), cancer pain, pain related to rheumathic arthritis, inflammatory pain, L-dopa-induced dyskinesias, dopaminomimetic-induced dyskinesias, L-dopa-induced dyskinesias in Parkinson's disease therapy, dopaminomimetic-induced dyskinesias in Parkinson's disease therapy, tardive dyskinesias, Parkinson's disease, anxiety disorders, panic disorders, anxiety and panic disorders, social anxiety disorder (SAD), generalized anxiety disorder, substance-induced anxiety disorder, eating disorders, obesity, binge eating disorders, Huntington's chorea, epilepsy, Alzheimer's disease, positive and negative symptoms of schizophrenia, cognitive impairment, functional gastrointestinal disorders, gastroesophageal reflux disease (GERD), migraine, irritable bowel syndrome (IBS), or for cognitive enhancement and/or neuroprotection.
b) For negative modulation of mGluR5: chronic pain, neuropathic pain, diabetic neuropathic pain (DNP), cancer pain, pain related to rheumathic arthritis, inflammatory pain, L-dopa-induced dyskinesias, dopaminomimetic-induced dyskinesias, L-dopa-induced dyskinesias in Parkinson's disease therapy, dopaminomimetic-induced dyskinesias in Parkinson's disease therapy, tardive dyskinesias, Parkinson's disease, anxiety disorders, panic disorders, anxiety and panic disorders, social anxiety disorder (SAD), generalized anxiety disorder, substance-induced anxiety disorder, eating disorders, obesity, binge eating disorders, migraine, irritable bowel syndrome (IBS), functional gastrointestinal disorders, gastroesophageal reflux disease (GERD), Huntington's chorea and/or epilepsy.
c) For positive modulation of mGluR5: Alzheimer's disease, positive and/or negative symptoms of schizophrenia, cognitive impairment, or for cognitive enhancement and/or neuroprotection.

The co-crystals of (6-bromo-pyrazolo[1,5-a]pyrimidin-2-yl)-(1(R)-methyl-3,4-dihydro-1H-isoquinolin-2-yl)-methanone according to the invention can especially be used for the treatment of binge eating disorders.

### BRIEF DESCRIPTION OF THE DRAWINGS

FIG. 1 is a powder X-ray diffraction chart of co-crystal of (6-bromo-pyrazolo[1,5-a]pyrimidin-2-yl)-(1(R)-methyl-3,4-dihydro-1H-isoquinolin-2-yl)-methanone and succinic acid according to Example 2a. Preparation: Powder as received according to Example 2a, 0.1 mm on Si (silicon).

FIG. 2 is a differential scanning calorimetry chart (DSC analysis chart) of co-crystal of (6-bromo-pyrazolo[1,5-a]pyrimidin-2-yl)-(1(R)-methyl-3,4-dihydro-1H-isoquinolin-2-yl)-methanone and succinic acid according to Example 2a. DSC measurement was carried out under nitrogen in closed Au crucibles with heating from −50.00° C. to 240° C. and heating rate of 10.00° C./min. (Peak=158.20° C.; Peak korr.=156.9° C., Peak height=17.6434 mW, Delta H=112.6 J/g).

FIG. 3 is a powder X-ray diffraction chart of co-crystal of (6-bromo-pyrazolo[1,5-a]pyrimidin-2-yl)-(1(R)-methyl-3,4-dihydro-1H-isoquinolin-2-yl)-methanone and gentisic acid according to Example 3a. Preparation: Powder as received according to Example 3a, 0.1 mm on Si (silicon).

FIG. 4 is a powder X-ray diffraction chart of co-crystal of (6-bromo-pyrazolo[1,5-a]pyrimidin-2-yl)-(1(R)-methyl-3,4-dihydro-1H-isoquinolin-2-yl)-methanone and gentisic acid according to Example 3c. Preparation: Powder as received according to Example 3c, 0.1 mm on Si (silicon).

FIG. 5 is a differential scanning calorimetry chart (DSC analysis chart) of co-crystal of (6-bromo-pyrazolo[1,5-a]pyrimidin-2-yl)-(1(R)-methyl-3,4-dihydro-1H-isoquinolin-2-yl)-methanone and gentisic acid according to Example 3c. DSC measurement was carried out under nitrogen in closed Au crucibles with heating from −50.00° C. to 250° C. and heating rate of 10.00° C./min. Melting Peak: Peak=148.17° C.; Peak korr.=147.4° C., Peak height=4.4950 mW, Area=185.679 mJ, Delta H=56.8696 J/g. Endothermic event: Peak=105.0° C.; Peak korr.=104.1° C., Peak height=0.1501 mW, Area=16.336 mJ, Delta H=5.0035 J/g.

FIG. 6 is a powder X-ray diffraction chart of co-crystal of (6-bromo-pyrazolo[1,5-a]pyrimidin-2-yl)-(1(R)-methyl-3,4-dihydro-1H-isoquinolin-2-yl)-methanone and xinafoic acid according to Example 4c. Preparation: Powder as received according to Example 4c, 0.1 mm on Si (silicon).

FIG. 7 is a differential scanning calorimetry chart (DSC analysis chart) of co-crystal of (6-bromo-pyrazolo[1,5-a]pyrimidin-2-yl)-(1(R)-methyl-3,4-d hydro-1H-isoquinolin-2-yl)-methanone and xinafoaic acid according to Example 4c. DSC measurement was carried out under nitrogen in closed Au crucibles with heating from −50.00° C. to 250° C. and heating rate of 10.00° C./min. Melting Peak: Peak=140.30° C.; Peak korr.=139.2° C., Peak height=10.2330 mW, Area=229.500 mJ, Delta H=79.3568 J/g. Endothermic event: Peak=202.9° C.; Peak korr.=202.6° C., Peak height=0.5661 mW, Area=67.712 mJ, Delta H=23.4134 J/g. Deposition: Peak=240.70° C.; Peak korr.=241.1° C., Peak height=−8.7764 mW, Area=−816.376 nil, Delta H=−282.2879 J/g.

The present invention is described in more detail by the following examples.

### EXAMPLES

### Example 1

### Characterisation of the Starting Material

The starting material of the pharmaceutically active ingredient (6-bromo-pyrazolo[1,5-a]pyrimidin-2-yl)-(1(R)-methyl-3,4-dihydro-1H-isoquinolin-2-yl)-methanone was prepared as described in WO 2008/015269.

The pKa value of (6-bromo-pyrazolo[1,5-a]pyrimidin-2-yl)-(1(R)-methyl-3,4-dihydro-1H-isoquinolin-2-yl)-methanone was calculated (using ACD/Labs) pKa DB v10.0 wherein pyrazolo(1,5-a)pyrimidine was used as correlation compound. The calculated pKa value of the protonated form is −1.97±0.30. The molecule therefore is a very weak base and the low pKa is not suitable for classical salt formation.

Additionally, the starting material was characterized, in particular by PXRD, FT-Raman and 1H NMR spectroscopy as described in Example 7.

The crystal structure of the free drug compound (6-bromo-pyrazolo[1,5-a]pyrimidin-2-yl)-(1(R)-methyl-3,4-dihydro-1H-isoquinolin-2-yl)-methanone was determined by X-ray crystallography. A crystal (colorless block, 0.16×0.32×0.36 mm) was measured on a Kappa APEX2 diffractometer at T=123K using graphite-monochromated molybdenum-K-alpha (Kα) radiation (wavelength λ=0.71073 Å (angstrom)) with θ max (Theta max)=36.345°. APEX2 Software suite has been used for data collection and Integration. The structure was solved by direct methods using the program SIR92. Least-squares refinement against F was carried out on all non-hydrogen atoms using the program CRYSTALS. The following crystal data have been found:

F(000)=752, orthorhombic, space group P 2₁2₁2₁, Z=4 calculated density D_{ca},,=1.605 mg m⁻³; a=7.5918(2) Å (angstrom), b=13.3879(4) Å (angstrom), c=15.1119(5) Å (angstrom), α (alpha)=90°, β (beta)=90°, γ (gamma)=90°, V=1535.95(8) Å³ (angstrom).

The co-crystal former 2,5-dihydroxybenzoic acid (gentisic acid, GEN) was purchased from Fluka (Order No. 37550, C₇H₆O₄; Ms, 154.12 g/mol). 1-Hydroxy-2-naphthoic acid (xinafoic acid, XIN) was purchased from Fluka (Order No. 55910; C₁₁H₈O₃; M_{w} 188.18 g/mol). Butanedioic acid (succinic acid, SUC) was purchased from Fluka (Order No. 14079, C₄H₆O₄; M_{w} 118.09 g/mol)

### Example 2

Preparation of co-crystals of (6-bromo-pyrazolo[1,5-a]pyrimidin-2-yl)-(1(R)-methyl-3,4-dihydro-1H-isoquinolin-2-yl)-methanone and succinic acid

### Example 2a

150 mg (6-bromo-pyrazolo[1,5-a]pyrimidin-2-yl)-(1(R)-methyl-3,4-dihydro-1H-isoquinolin-2-yl)-methanone and 57.8 mg succinic acid were mixed. 0.15 ml isopropyl ether was added. The mixture was stirred at room temperature for about 24 hours and finally the solvent was evaporated at room temperature in air (open vial). A white powder was obtained.

The obtained powder was characterised by FT Raman. The FT Raman spectrum shows a mixture of free active ingredient and succinic acid.

2 ml isopropyl ether was added to the residue of the obtained powder. The mixture was stirred for about 16 hours. The resulting solid was filtered off and dried in air. A colourless powder was obtained which was characterised by FT Raman, PXRD, ¹H-NMR, TG-FTIR, DSC, DVS as described in Example 7.

The obtained crystalline powder showed a unique Raman spectrum, NMR data confirmed the given co-crystalline structure.

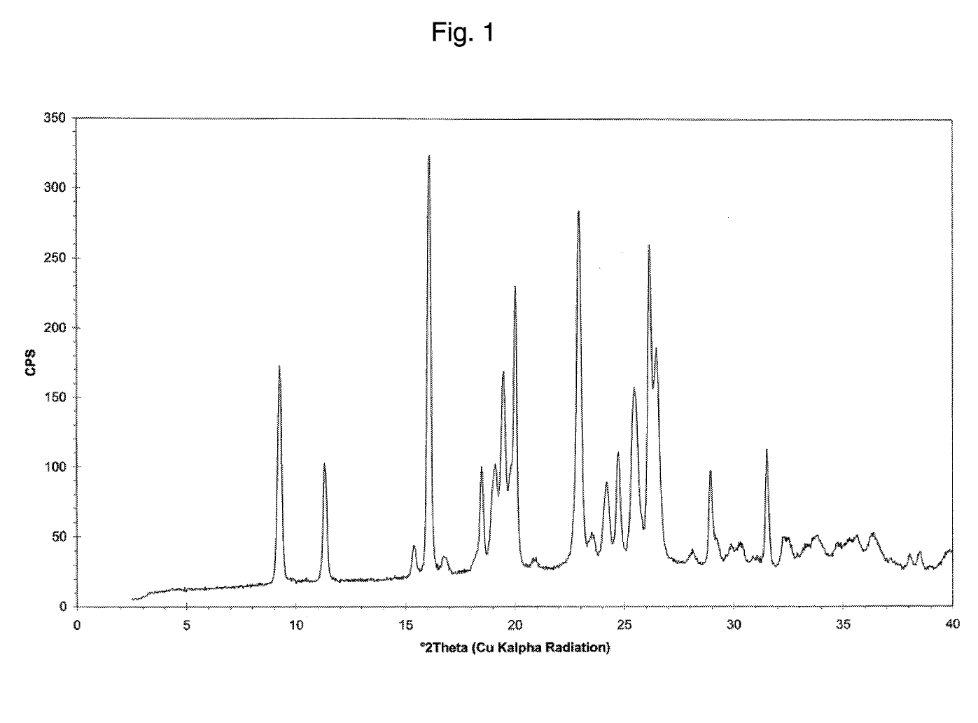

The powder was characterised by a PXRD pattern which is shown in FIG. 1. DVS data demonstrated that the obtained crystalline powder does not uptake water (not hygroscopic).

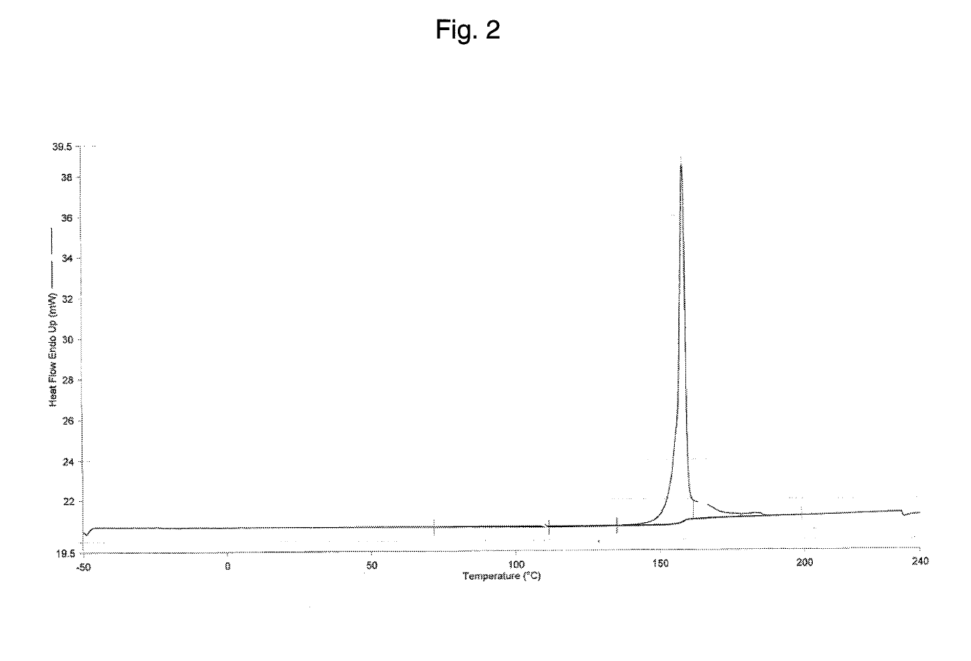

TG-FTIR measurements demonstrate that the obtained crystalline powder contained traces of isopropyl ether/water and a degradation above 150° C. Furthermore, the obtained crystalline powder was characterised by a melting peak at 156.9° C. (DSC). The DSC diagram is shown in FIG. 2.

### Example 2b

150 mg (6-bromo-pyrazolo[1,5-a]pyrimidin-2-yl)-(1(R)-methyl-3,4-dihydro-1H-isoquinolin-2-yl)-methanone was dissolved in 5 ml acetone. 57.8 mg succinic acid (dissolved in 2 ml acetone) was added. The solvent was evaporated at room temperature under nitrogen flow without flow control. Colourless powder was obtained. The obtained powder was characterised by FT Raman as described in Example 7 wherein the Raman spectrum is identically with free base.

2 ml 2-propanol was added to the residue obtained. The mixture was stirred for about 16 hours. The resulting solid was filtered off and dried in air. Colourless powder was obtained which was characterised by FT Raman. The co-crystal shows the same Raman spectrum as co-crystal according to example 2a.

### Example 3

Preparation of co-crystal of (6-bromo-pyrazolo[1,5-a]pyrimidin-2-yl)-(1(R)-methyl-3,4-dihydro-1H-isoquinolin-2-yl)-methanone and 2,5-dihydroxybenzoic acid (gentisic acid)

### Example 3a

150 mg (6-bromo-pyrazolo[1,5-a]pyrimidin-2-yl)-(1(R)-methyl-3,4-dihydro-1H-isoquinolin-2-yl)-methanone was dissolved in 5 ml acetone. 75.5 mg gentisic acid (dissolved in 2 ml acetone) was added. The solvent was evaporated at room temperature under nitrogen flow without flow control. Ivory colored powder was obtained.

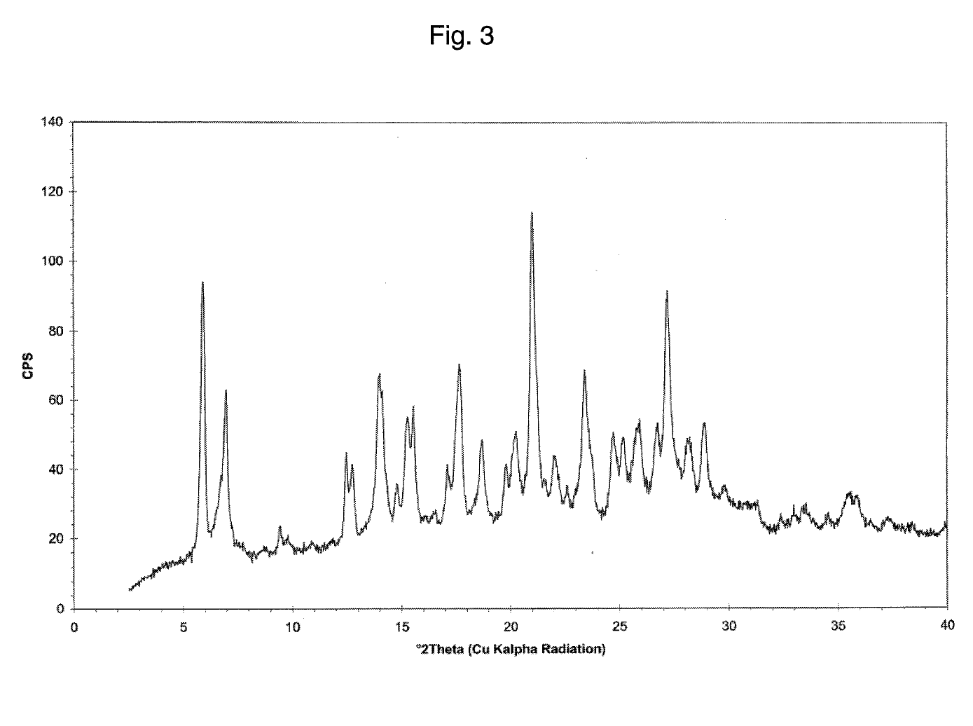

The obtained product was characterised by FT Raman, PXRD (FIG. 3), ¹H-NMR as described in example 7.

The X-ray diffraction pattern of obtained powder is shown on FIG. 3 and confirms a crystalline form (co-crystal). ¹H-NMR spectrum confirmed the given structure of a co-crystal.

### Example 3b

150 mg of (6-bromo-pyrazolo[1,5-a]pyrimidin-2-yl)-(1(R)-methyl-3,4-dihydro-1H-isoquinolin-2-yl)-methanone and 75.5 mg gentisic acid were mixed. 0.15 ml isopropyl ether was added. The mixture was stirred at room temperature for about 24 hours and finally the solvent was evaporated at room temperature in air (open vial). An ivory colored powder was obtained. FT Raman spectrum of obtained powder agrees with FT Raman spectrum of Example 3a).

### Example 3c

150 mg of (6-bromo-pyrazolo[1,5-a]pyrimidin-2-yl)-(1(R)-methyl-3,4-dihydro-1H-isoquinolin-2-yl)-methanone was dissolved in 5 ml acetone. 62.3 mg gentisic acid was added. The solvent was evaporated at room temperature under nitrogen flow without flow control. Ivory colored powder was obtained.

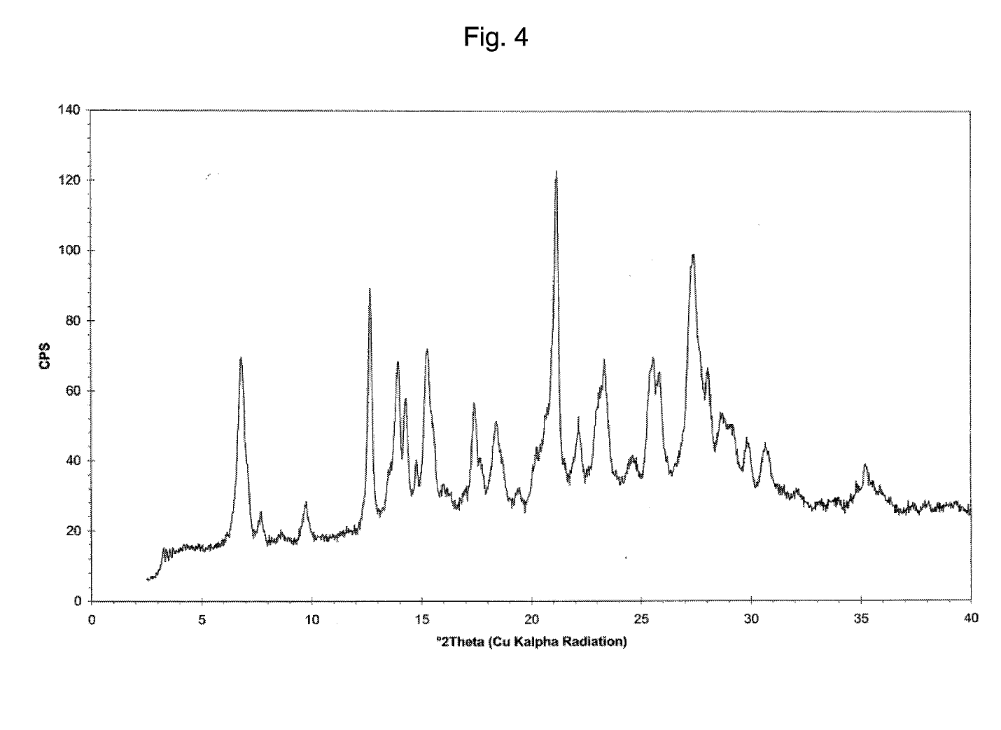

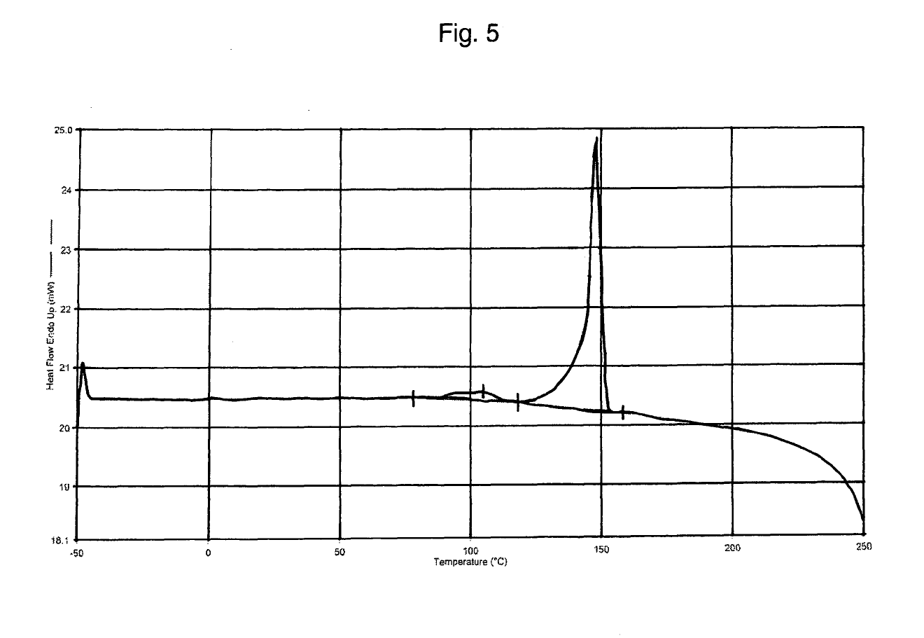

The obtained product was characterised by FT Raman, PXRD (FIG. 4), ¹H-NMR, TG-FTIR, DSC (FIG. 5), DVS, and FT Raman spectroscopy as described in Example 7.

The PXRD pattern as seen in FIG. 4 shows a crystalline structure different from X-ray pattern of co-crystal according to example 3a. Thus, another polymorphic form of co-crystal was obtained. Further it is demonstrated that the obtained co-crystal contains traces of isopropyl ether/acetone. NMR data agrees with given structure.

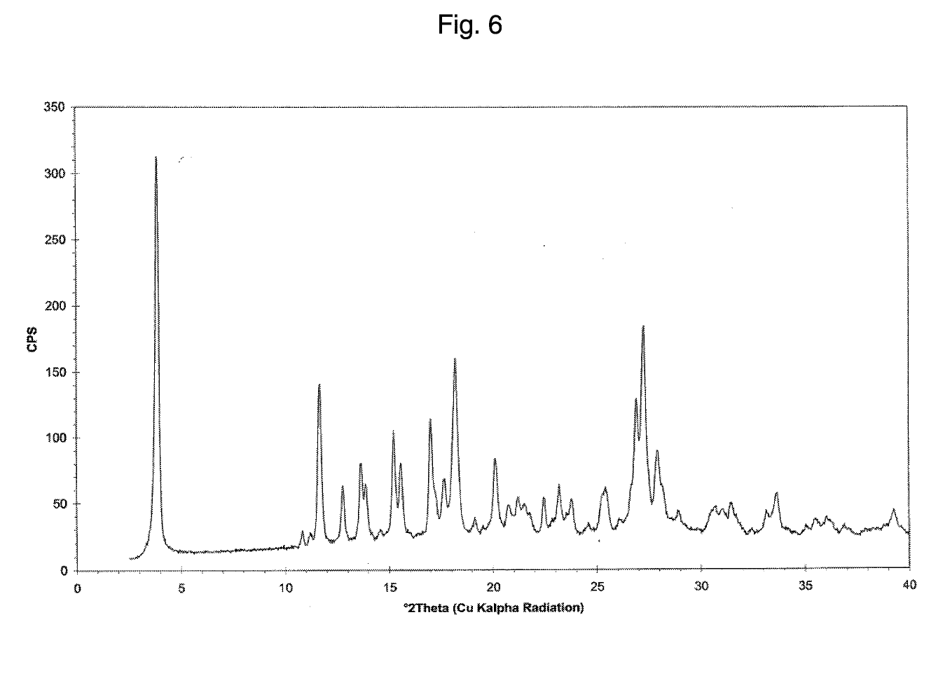

The TG-FTIR analysis shows degradation above 150° C.; DSC diagram shows endothermic event at 104° C., melting peak at 147° C. (see DSC diagram FIG. 6)

As a result from DVS measurements, the obtained co-crystal exhibits minimal water uptake and is not hygroscopic.

### Example 4

Preparation of co-crystal of (6-bromo-pyrazolo[1,5-a]pyrimidin-2-yl)-(1(R)-methyl-3,4-dihydro-1H-isoquinolin-2-yl)-methanone and 1-hydroxy-2-naphthoic acid (xinafoic acid)

### Example 4a

150 mg (6-bromo-pyrazolo[1,5-a]pyrimidin-2-yl)-(1(R)-methyl-3,4-dihydro-1H-isoquinolin-2-yl)-methanone was dissolved in 5 ml EtOAc. 92.1 mg 1-hydroxy-2-naphthoic acid (xinafoic acid) (dissolved in 2 ml ethyl acetate) was added. The solvent was evaporated at room temperature under nitrogen flow without flow control. Ivory colored powder was obtained which was characterised by FT Raman, PXRD, ¹H-NMR as described in Example 7.

X-ray diffraction pattern of obtained powder is extensively identical to PXRD pattern according to Example 4c and confirms a crystalline form (co-crystal). ¹H-NMR spectrum agrees with expected structure of a co-crystal.

### Example 4b

150 mg 6-bromo-pyrazolo[1,5-a]pyrimidin-2-yl)-(1(R)-methyl-3,4-dihydro-1H-isoquinolin-2-yl)-methanone and 92.1 mg 1-hydroxy-2-naphthoic acid were mixed. 0.15 ml isopropyl ether was added. The mixture was stirred at room temperature (r.t.) for about 24 hours and finally the solvent was evaporated at room temperature (r.t.) in air (open vial). Ivory coloured powder was obtained wherein the Raman Spectrum of obtained powder is a mixture of co-crystal and active ingredient.

2 ml isopropyl ether was added to the obtained residue. The mixture was stirred for about 16 hours. The resulting solid was filtered off and dried in air. Ivory coloured powder was obtained. FT Raman of obtained powder agrees with spectrum of co-crystal obtained according to example 4a.

### Example 4c

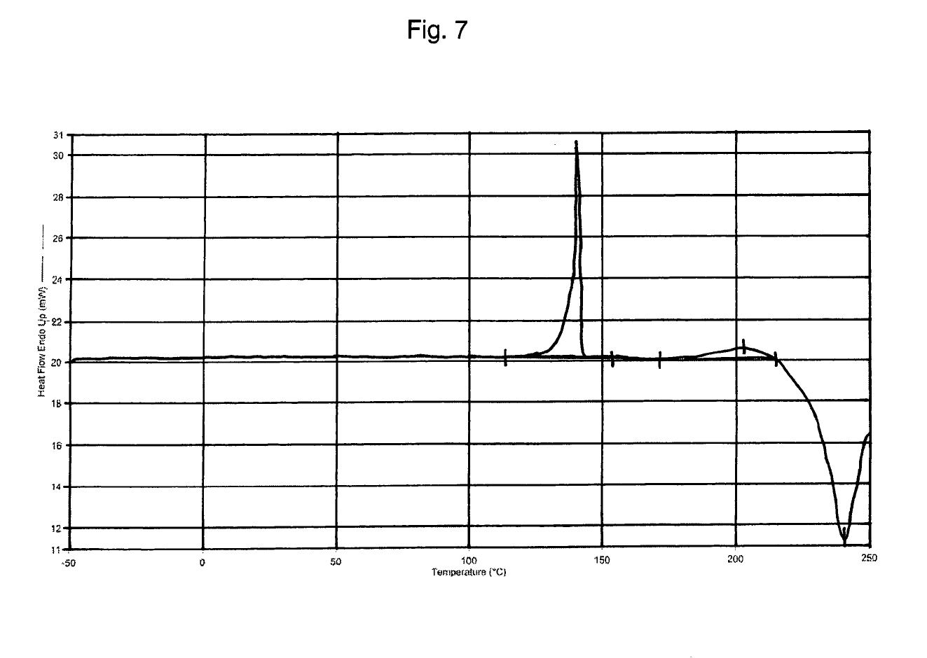

150 mg 6-bromo-pyrazolo[1,5-a]pyrimidin-2-yl)-(1(R)-methyl-3,4-dihydro-1H-isoquinolin-2-yl)-methanone was dissolved in 5 ml EtOAc. 76 mg 1-hydroxy-2-naphthoic acid (dissolved in 2 ml EtOAc) was added. The solvent was evaporated at room temperature (r.t.) under nitrogen flow without flow control. An ivory colored powder was obtained which was characterised by FT Raman, PXRD (FIG. 6), ¹H NMR, TG-FTIR, DSC (FIG. 7), DVS as described in Example 7.

FT Raman of obtained powder agrees with spectrum of co-crystal obtained according to example 4a.

X-ray diffraction pattern of obtained powder is shown on FIG. 6 and confirms a crystalline form (co-crystal). ¹H-NMR spectrum agrees with given structure of a co-crystal.

DSC analysis (DSC diagram is shown in FIG. 7) shows that the obtained co-crystals exhibits a melting peak at 139° C., and exothermic event at 241° C. TG-FTIR measurement demonstrates that xinafoic acid co-crystal contains traces of ethyl acetate and shows degradation above 150° C.

DVS measurements shows reversible water uptake above 80% relative humidity (r.h.). Thus, the obtained co-crystals of known compound A and xinafoic acid are slightly hygroscopic.

### Example 5

### Determination of Aqueous Solubility

Each co-crystal (according to example 2a, 3c; and 4c) was suspended in water. The samples were shaken with a temperature controlled “Thermomixer comfort” from Eppendorf at 800 rpm (24 hours, 23° C.). The resulting suspensions were filtered with Millipore Centrifugal Filter Device UFC30VVNB (0.1) and Centrifuge Hettich EBA 12 R (10.000 g). The obtained solids were characterized by FT-Raman spectroscopy and compared with the spectrum before solubility test, wherein no change in form was observed.

The pH of the filtrate was measured, and the concentration of the free base was determined by HPLC (method is described in Example 7).

The aqueous solubility of the free drug compound which is near of the detection limit was determined as well (same conditions) and represents below 10 μg/ml (below 0.01 mg/ml) at pH 8.5.

The measured solubility of the novel co-crystals are summarized in Table 1.

TABLE 1
Solubility of co-crystals determined by HPLC

| | Solubility | |
|---|---|---|
| sample | [mg/ml] | pH |
| Example 2a (co- | 0.02 | 3.1 |
| crystal with succinic acid) | | |
| Example 3c (co- | 0.03 | 3.0 |
| crystal with gentic acid) | | |
| Example 4c (co- | 0.04 | 5.2 |
| crystal with xinafoic acid) | | |

The co-crystals according to the present invention surprisingly have at least a two-fold higher solubility than the free drug.

### Example 6

### Characterisation of Co-Crystals of Succinic Acid, Gentisic Acid, and Xinafoic Acid by Thermal Analytical Techniques

The following co-crystals of (6-bromo-pyrazolo[1,5-a]pyrimidin-2-yl)-(1(R)-methyl-3,4-dihydro-1H-isoquinolin-2-yl)-methanone was characterised by TG-FTIR, DSC and DVS as described in example 7:
co-crystal of succinic acid according to example 2a,
co-crystal of gentisic acid according to example 3c, and
co-crystal of xinafoic acid according to example 4c.

### Example 6a

TG-FTIR was performed on the samples as mentioned above. The results are summarized in Table 1 below 1.

TABLE 1
TG-FTIR experiments

| Sample | Event | Comments |
|---|---|---|
| Example 2a | mass loss, 30-150° C., 0.2% | traces of isopropyl |
| (succinic acid | | ether |
| co-crystal) | mass loss, 150-250° C., 4.7% | decomposition |
| Example 3c | mass loss, 50-180° C., 1.0% | traces of acetone |
| (gentisic acid | mass loss, 180-250° C, 2.7% | decomposition |
| co-crystal) | | |
| Example 4c | mass loss, 50-160° C., 0.5% | traces of ethyl |
| (xinafoic acid | | acetate |
| co-crystal) | mass loss, 160-250° C., 9.0% | decomposition |

The succinic acid co-crystal sample contains traces of isopropyl ether (solvent used for preparation). Above 150° C. degradation was observed.

The gentisic acid co-crystal sample contains traces of acetone (solvent used for preparation). Above 180° C. degradation was observed.

The xinafoic acid co-crystal sample contains traces of ethyl acetate (solvent used for to preparation). Above 160° C. degradation was observed.

### Example 6b

DSC was performed on the samples as mentioned above. The results are summarized in Table 2 below.

TABLE 2
DSC experiments

| Sample | Event | Comments |
|---|---|---|
| Example 2a | endothermic event, 156.9° C., | melting peak |
| (succinic acid | ΔH = 113 J/g | |
| co-crystal) | | |
| Example 3c | broad endothermic event, | traces of the starting |
| (gentisic acid | 104.1° C., ΔH = 5 J/g | materials or other |
| co-crystal) | | impurities |
| | endothermic event, 147.4° C., | melting peak |
| | ΔH = 57 J/g | |
| Example 4c | endothermic event, 139.2° C., | melting peak |
| (xinafoic acid | ΔH = 79 J/g | |
| co-crystal) | exothermic event, >200° C. | degradation |

Compound A melts in the range of 132 to 140° C. (peak 132.9° C.).

The DSC of the succinic acid co-crystal according to Example 2a (see FIG. 2) shows a sharp melting peak at 156.9° C.

DSC of the gentisic acid co-crystal according to Example 3c (see FIG. 5) shows a broad endothermic event at 104° C.=5 J/g). Probably the sample contains traces of the starting materials and/or other impurities. A second endothermic event at 147° C. can be attributed to the melting of the co-crystal. Degradation was observed above 200° C.

The DSC of the xinafoic acid co-crystal according to Example 4c (see FIG. 7) shows a sharp melting peak at 139.2° C. and degradation above 200° C.

### Example 6c

DVS (50%->0%->95%->50% r.h.) was performed on the samples as mentioned above. The results are summarized in the following:

The DVS of the gentisic acid co-crystal (Example 2a) shows only minimal and reversible mass changes over the tested humidity range. A mass change Δm (change of relative humidity (r.h.) from 50 to 85%) of about 0.1% was observed, the co-crystal is not hygroscopic. The post-DVS Raman spectrum does not indicate any change in form.

The DVS of the succinic acid co-crystal (Example 3c) shows only minimal and reversible mass changes over the tested humidity range. A mass change Δm (change of relative humidity (r.h.) from 50 to 85%) of about 0.1% was observed, the co-crystal is not hygroscopic. The post-DVS Raman spectrum does not indicate any change in form. The DVS of the xinafoic acid co-crystal (Example 4c) shows a reversible water uptake above 80% r.h. with hysteresis. A mass change Δm (change of relative humidity (r.h.) from 50 to 85%) of about 1% was observed. The co-crystal is slightly hygroscopic. More water was taken up as the humidity was increased to 95% r.h. (approximately 2 wt.-% total water content, equilibrium reached). Upon lowering the relative humidity again, the water content decreased and reverted to the original mass. The post-DVS Raman spectrum does not indicate any change in form.

### Example 7

### Instrumental Measurement Conditions

### Example 7a

DSC (differential scanning calorimetry)/Perkin Elmer DSC 7 was used with closed Au crucibles, heating rate: 10 or 20° C./Min, range: −50° C. to 250° C.

### Example 7b

### DVS (Dynamic Vapour Sorption)

Surface Measurement Systems Ltd. DVS-1 water vapour sorption analyzer and Projekt Messtechnik SPS 11-100n multi-sample water vapor sorption analyzer were used.

The sample was allowed to equilibrate at 50% relative humidity (r.h.) before starting e.g. the following predefined humidity program:
2 h at 0% r.h.
0 to 95% r.h. (5%/h)
3 h at 95% r.h.
95 to 0% (10%/h)
2 h at 0% r.h.

### Example 7c

The used HPLC (High Performance liquid chromatography) system is characterised as follows:

| | |
|---|---|
| equipment: | TSP HPLC (UV3000, AS3000, |
| | P4000, SCM1000 Soft. Version 4.1) |
| Column: | Waters, XTerra MS C18 4.6 × |
| | 100 mm 5μ (CC01) |
| mobile phase A | distilled H2O + 0.1% TFA |
| mobile phase B | MeCN + 0.1% TFA |
| reference concentration | ca. 0.04 mg/ml |
| retention time | 13.1 min |
| gradient | 0.0 min 95% A/5% B |
| | 20.0 min 5% A/95% B |
| | 21.0 min 95% A/5% B |
| | 30.0 min 95% A/5% B |
| flow | 1.0 ml/min |
| injection volume | 10 μl |
| wavelength | 254 nm. |

### Example 7d

### NMR (Nuclear Magnetic Resonance)

The ¹H NMR spectra were recorded at 300.13 MHz on Bruker DPX300 instrument.

### Example 7e

### Raman Microscopy

The Raman spectra were recorded at Renishaw RM 1000 with a stabilized diode laser 785-nm excitation and NIR-enhanced Peltier-cooled CCD camera as detector. Measurements were carried out with a long working distance 20× objective. Measurement range 2000-100 cm⁻¹.

### Example 7f

### FT-Raman Spectroscopy (Fourier Transform Raman Spectroscopy)

The FT-Raman spectra were recorded at Bruker RFS100 with Nd:YAG 1064 nm excitation, 100 mW laser power and a Ge detector, 64 scans, range 25-3500 cm-1, 2 cm-1 resolution.

### Example 7 g

### TG-FTIR (Thermogravimetry Coupled with Fourier Transformed Infrared Spectroscopy)

TG-FTIR was carried out with Netzsch Thermo-Microbalance TG 209 with Bruker FT-IR Spectrometer, Vector 22 in an Al crucible (open or with microhole), N2 atmosphere, heating rate 10° C. min⁻¹, range 25-250° C.

### Example 7h

### Solubility Determination

Suspension of co-crystal in water was agitated with a temperature controlled. Thermomixer comfort” from Eppendorf with 800 rpm (24 hours, 23° C.). The suspension was filtered with Millipore Centrifugal Filter Device UFC30VVNB (0.1μ) and Centrifuge Hettich EBA 12 R (10,000 g).

### Example 7i

### PXRD (Powder X-Ray Diffraction)

Powder X-ray diffraction patterns were recorded at Bruker D8 with Copper Ka radiation (Cu-Kα₁, wavelength λ=1.540598 Å (angstrom)), 40 kV/40 mA, and LynxEye detector, 0.02°2θ step size, 37 s step time.

Sample preparation: The samples were generally measured without any special treatment other than the application of slight pressure to get a flat surface. Silicon single crystal sample holder types: a) standard holder for polymorphism screening, 0.1 mm deep, less than 20 mg sample required; b) 0.5 mm deep, 12 mm cavity diameter for c. 40 mg; c) 1.0 mm deep, 12 mm cavity diameter for c. 80 mg. All samples measured on the Bruker D8 are rotated during the'measurement.

### Example 8

### Determination of Dissolution Rate

For each co-crystal (according to example 2a 3c and 4c) and for the free drug compound (6-bromo-pyrazolo[1,5-a]pyrimidin-2-yl)-(1(R)-methyl-3,4-dihydro-1H-isoquinolin-2-yl)-methanone) dissolution of a compressed tablet was monitored in 0.15 M aqueous potassium chloride, using UV-absorption spectroscopy, as the pH was increased, through four sectors, to simulate passage of the tablet through the gastrointestinal tract (sector 1: pH 2.0; sector II: pH 3.9; sector III: pH 5.4; sector IV: pH 7.3).

The tablet was compressed under a weight of approximately 50000 pounds per square inch, and had a diameter of 3 mm. Only one face of the tablet was exposed to the dissolution medium, which contained an acetate/phosphate buffer system to minimise perturbation of the experimental pH from dissolution of the drug. Stirring of the solution was continuous and at a constant rate. The absorption data was converted to absolute sample weights using previously determined, pH-dependent, molar extinction coefficients. An appropriate wavelength range was chosen to ensure that spectroscopic data with an absorption value of <1.3 was analyzed, avoiding erroneous dissolution results due to saturation of the UV light source. Dissolution rates were calculated from the fit of a first-order exponential equation to the experimental data obtained.

The dissolution rates listed in the Table 3 were determined at pH 2.0, 3.9, 5.4, and 7.3 (+/−0.1), at a temperature of 23° C. (+/−1° C.). In case that no data given in the Table 3 it was found, that the dissolution rate did not change significantly in moving from the second to the third and fourth sectors.

TABLE 3
Dissolution rates

Dissolution rate
[μg min⁻¹]
pH

| Sample | 2.0 | 3.9 | 5.4 | 7.3 |
|---|---|---|---|---|
| Free drug compound | 0.58 | 0.22 | — | — |
| (6-bromo-pyrazolo[1,5- | | | | |
| a]pyrimidin-2-yl)-(1(R)- | | | | |
| methyl-3,4-dihydro-1H- | | | | |
| isoquinolin-2-yl)-methanone) | | | | |
| Example 2a | 2.06 | 1.58 | — | — |
| (succinic acid co-crystal) | | | | |
| Example 3c | 2.04 | 1.06 | — | — |
| (gentisic acid co-crystal) | | | | |
| Example 4c | 0.46 | 0.68 | 1.26 | 1.44 |
| (xinafoic acid co-crystal) | | | | |

For instance, in the case of the succinate co-crystal of MRZ 8456 a 3-fold enhancement of the dissolution rate was observed compared to the free drug MRZ 8456 at pH 2.

1. A co-crystal of (6-bromo-pyrazolo[1,5-a]pyrimidin-2-yl)-(1-methyl-3,4-dihydro-1H-isoquinolin-2-yl)-methanone and a co-crystal former, wherein the co-crystal former is a carboxylic acid of general formula (I) 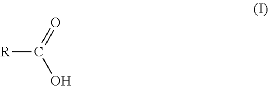
wherein R denotes: 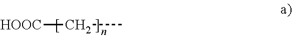
wherein n is 1,2,3, or 4, 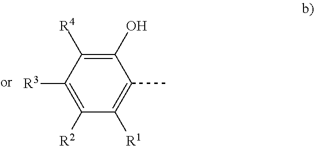
wherein
R¹ and R² are independently from each other hydrogen, hydroxyl or carboxyl,
R³ and R⁴ are independently from each other hydrogen, hydroxyl or carboxyl,
or R³ and R⁴, together with the carbon atoms carrying them, form an aromatic six-membered ring which may be substituted by one to four groups selected from C₁-C₅ alkyl, hydroxyl, and carboxyl.

2. The co-crystal according to claim 1, wherein the co-crystal former is a carboxylic acid of general formula I 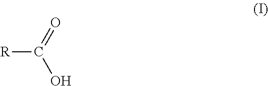
wherein R denotes: 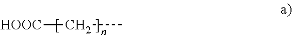
wherein n is 2 or 3, 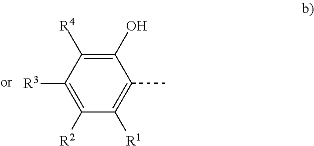
wherein
R¹ and R² are independently from each other hydrogen or hydroxyl,
R³ and R⁴ are hydrogen,
or R³ and R⁴, together with the carbon atoms carrying them, ray also form an unsubstituted aromatic six-membered ring.

3. The co-crystal according to claim 1, wherein the co-crystal is a co-crystal of (6-bromo-pyrazolo[1,5-a]pyrimidin-2-yl)-(1(R)-methyl-3,4-dihydro-1H-isoquinolin-2-yl)-methanone and a co-crystal former, wherein the co-crystal former is a carboxylic acid selected from the group consisting of gentisic acid, succinic acid and xinafoic acid.

4. The co-crystal according to claim 1, wherein the molar ratio of (6-bromo-pyrazolo[1,5-a]pyrimidin-2-yl)-(1-methyl-3,4-dihydro-1H-isoquinolin-2-yl)-methanone: co-crystal former is in the range from 1:0.1 to 1:10.

5. The co-crystal according to claim 1, wherein the co-crystal is a co-crystal of (6-bromo-pyrazolo[1,5-a]pyrimidin-2-yl)-(1(R)-methyl-3,4-dihydro-1H-isoquinolin-2-yl)-triethanone and a co-crystal former, wherein the co-crystal former is succinic acid, and wherein the co-crystal is characterised by:
at least two powder X-ray diffraction (PXRD) peaks selected from the group consisting of 9.3, 16.0, 20.0, 22.9, and 26.0 degrees two-theta (°2θ)+/−0.3 degrees two-theta (° 2θ),
melting peak at about 156.9° C. measured by differential scanning calorimetry (DSC).

6. The co-crystal according to claim 1, wherein the co-crystal is a co-crystal of (6-bromo-pyrazolo[1,5-a]pyrimidin-2-yl)-(1(R)-methyl-3,4-dihydro-1H-isoquinolin-2-yl)-methanone and a co-crystal former, wherein the co-crystal former is gentisic acid, and wherein the co-crystal is characterised by
at least four powder X-ray diffraction (PXRD) peaks selected from the group consisting of 6.0, 7.0, 14.0, 17.6, 21.0, 23.4, and 27.2 degrees two-theta (°2θ)+/−0.3 degrees two-theta (°2θ).

7. The co-crystal according to any of claim 1, wherein the co-crystal is a co-crystal of (6-bromo-pyrazolo[1,5-a]pyrimidin-2-yl)-(1(R)-methyl-3,4-dihydro-1H-1-isoquinolin-2-yl)-methanone and a co-crystal former, wherein the co-crystal former is gentisic acid, and wherein the co-crystal is characterised by:
at least one powder X-ray diffraction (PXRD) peaks selected from the group consisting of 6.9, 12.6, 21.2, and 27.5 degrees two-theta (°2θ)+/−0.3 degrees two-theta (°2θ),
melting peak at about 147.4° C. measured by differential scanning calorimetry (DSC).

8. The co-crystal according to claim 1, wherein the co-crystal is a co-crystal of (6-bromo-pyrazolo[1,5-a]pyrimidin-2-yl)-(1(R)-methyl-3,4-dihydro-1H-isoquinolin-2-yl)-methanone and a co-crystal former, wherein the co-crystal former is xinafoic acid and wherein the co-crystal is characterised by:
at least one powder X-ray diffraction (PXRD) peaks selected from the group consisting of 3.9, 11.6, 18.1, and 27.2 degrees two-theta (°2θ)+/−0.3 degrees two-theta (°2θ),
melting peak at about 139.2° C. measured by differential scanning calorimetry (DSC).

9. A pharmaceutical composition comprising the co-crystal according to claim 1 as an active ingredient together with one or more pharmaceutically acceptable excipients.

10. A method of treating and/or preventing a condition or disease associated with abnormal glutamate neurotransmission in a mammal, comprising administering to the mammal a therapeutically effective amount of the co-crystal according to claim 1.

11. The method according to claim 10, wherein the condition or disease is Alzheimer's disease, positive and/or negative symptoms of schizophrenia, cognitive impairment, or cognitive enhancement and/or neuroprotection.

12. A method for the preparation of a co-crystal of (6-bromo-pyrazolo[1,5-a]pyrimidin-2-yl)-(1(R)-methyl-3,4-dihydro-1H-isoquinolin-2-yl)-methanone and a co-crystal former, wherein the co-crystal former is a carboxylic acid of general formula I 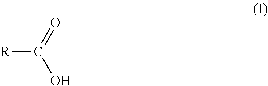
wherein R denotes: 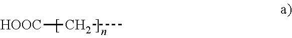
where n is 1,2,3, or 4, 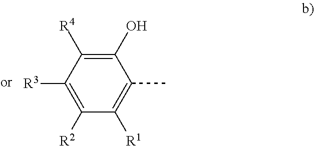
where
R¹ and R² are independently from each other hydrogen, hydroxyl or carboxyl,
R³ and R⁴ are independently from each other hydrogen, hydroxyl or carboxyl,
or R³ and R⁴, together with the carbon atoms carrying them, form an aromatic six-membered ring which may be substituted by one to four groups selected from C₁-C₅ alkyl, hydroxyl, and carboxyl,
comprising the following steps:
a) dissolving 6-bromo-pyrazolo[1,5-a]pyrimidin-2-yl)-(1(R)-methyl-3,4-dihydro-1H-isoquinolin-2-yl)-methanone and the co-crystal former in a solvent S1,
b) evaporation of solvent S1,
c) optionally dispersing the residue obtained in step b) in a solvent S2 for at least 10 h in a slurry.

13. The method for the preparation of the co-crystal according to claim 12, wherein the co-crystal former is a carboxylic acid selected from the group consisting of gentisic acid, succinic acid, and xinafoic acid, comprising the following steps:
a) dissolving 6-bromo-pyrazolo[1,5-a]pyrimidin-2-yl)-(1(R)-methyl-3,4-dihydro-1H-isoquinolin-2-yl)-methanone and the co-crystal former in a solvent S1,
b) evaporation of solvent S1,
c) optionally dispersing the residue obtained in step b) in a solvent S2 for at least 10 h in a slurry.

14. The method for the preparation of the co-crystal according to claim 12, wherein the solvents S1 and optionally S2 are at least one solvent selected from the group consisting of acetone, 1-butanol, tert-butyl-methyl ether (TBME), dimethyl sulfoxide (DMSO), ethanol, ethyl acetate, methyl ethyl ketone (MEK), 1-propanol, 2-propanol, tetrahydrofuran (THE), acetonitrile, dichloro methane, N,N-dimethyl formamide (DMF), 1-octanol, methanol, toluene, water, isopropyl ether (IPE), and N-methyl pyrrolidone (NMP).
